(19) United States
(12) Patent Application Publication
(10) Pub. No.: US 2026/0224368 A1
(43) Pub. Date: Aug. 6, 2026

(54) BIPOLAR PROSTHESES SUBASSEMBLY AND BIPOLAR PROSTHESES ASSEMBLY

(71) Applicants: Smith & Nephew Asia Pacific Pte. Limited, Singapore (SG); Smith & Nephew Orthopaedics AG, Zug (CH); Smith & Nephew, Inc., Memphis, TN (US)

(72) Inventors: Xinliang Wang, Beijing (CN); Hongli Shi, Beijing (CN); Choon Hong SIM, Singapore (SG)

(73) Assignees: Smith & Nephew Asia Pacific Pte. Limited, Singapore (SG); Smith & Nephew Orthopaedics AG, Zug (CH); Smith & Nephew, Inc., Memphis, TN (US)

(21) Appl. No.: 19/118,968

(22) PCT Filed: Oct. 28, 2022
(86) PCT No.: PCT/CN2022/128260

§ 371 Date: Apr. 7, 2025

(51) Int. Cl. A61F 2/34; A61F 2/30; A61F 2/32

(52) U.S. Cl. CPC A61F 2/34; A61F 2/3094; A61F 2002/30495; A61F 2002/305; A61F 2002/30822; A61F 2002/3241

(57) ABSTRACT

A bipolar prosthesis subassembly (100) is provided. The bipolar prosthesis subassembly (100) comprises: an outer shell (10); an inner liner (20) configured to dispose inside of the outer shell (10) in a manner of non-interference fit, the inner liner (20) is provided with a circumferential groove (24); and a split lock ring (30) configured to elastically snap into the circumferential groove (24) of the inner liner (20).

### TECHNICAL FIELD

The present invention relates to surgical implants for replacing a femoral head of a hip joint, and in particular, to a bipolar prosthesis subassembly and a bipolar prosthesis assembly including the bipolar prosthesis subassembly.

### BACKGROUND

Bipolar head prostheses are surgical implants used in combination with a femoral stem for hip replacement surgery to replace the femoral head of the hip joint.

The bipolar prosthesis generally consists of an outer shell and an inner liner. The inner liner fits in the outer shell in a manner of interference fit. The femoral head is held in the inner liner with a locking ring. However, as the rigidity of the inner liner is weaker than that of the outer shell, the inner liner may be deformed during the assembly of the bipolar prosthesis, thus affecting the dimensional accuracy and roundness of the inner liner, which in turn increases the wear between the inner liner and the femoral head, resulting in a large amount of fragments leading to loosening of the bipolar prosthesis.

In addition, the bipolar prosthesis is not easy to install, at first, the lock ring is placed in the outer groove of the femoral head, and the femoral head with the lock ring is placed inside the inner liner, then the inner liner with the femoral head locked in place is inserted into the outer shell with a large thrust. The bipolar prosthesis entails several steps to assembly.

Thus, it is desirable to develop a bipolar prosthesis with reduced deformation and wear, which is easy to assembly.

### SUMMARY OF THE INVENTION

Objects of the present disclosure are to provide a bipolar prosthesis assembly with reduced deformation and wear, which is easy to assemble.

In one aspect, a bipolar prosthesis subassembly is provided. The bipolar prosthesis subassembly comprises:
an outer shell;
an inner liner configured to dispose inside of the outer shell in a manner of non-interference fit, the inner liner is provided with a circumferential groove; and
a split lock ring configured to elastically snap into the circumferential groove of the inner liner,
wherein the inner liner defines a sphere-shaped cavity defining an equator, the circumferential groove of the inner liner extends from a first position below and in proximity to the equator to a second position above the equator and tapers from the first position to the second position, the split lock ring is movable in the circumferential groove between an assembly position and a locking position along the longitudinal axis, in the assembly position, the split lock ring can be expanded to an inner diameter lager than an outer diameter of a femoral head so that the femoral head can be inserted into the sphere-shaped cavity, in the locking position, the split lock ring elastically returns to an inner diameter less than the outer diameter of the femoral head so as to securely hold the femoral head in place inside the inner liner.

In other aspects, the outer shell includes a sphere-shaped outer surface and an inner surface with a first distal inner surface portion of sphere-shape and a second proximal inner surface portion of cylindrical shape, the inner liner includes a sphere-shaped inner surface and an outer surface with a first distal outer surface portion of sphere-shape and a second proximal outer surface portion of cylindrical shape.

In other aspects, the outer shell is provided with a circumferential locking groove on the second proximal inner surface portion, the inner liner is provided with a circumferential distal locking protrusion configured to snap into the circumferential locking groove of the outer shell to secure the inner liner within the outer shell.

In other aspects, the circumferential distal locking protrusion is sized less than the circumferential locking groove of the outer shell, so as to form a space between the circumferential distal locking protrusion and the circumferential locking groove of the outer shell.

In other aspects, the proximal end of the inner liner extends beyond the sphere-shaped outer surface of the outer shell along a proximal direction, and is provided with a partial sphere-shaped transition portion, which forms smooth transition to the sphere-shaped outer surface of the outer shell.

In other aspects, the split lock ring is provided with a circumferential groove at outer surface thereof, the bipolar prosthesis subassembly further comprising a split reinforcing ring configured to elastically snap into the circumferential groove of the split lock ring.

In other aspects, the split lock ring is made of a plastic material, the split reinforcing ring is made of a metal or metal alloy.

In other aspects, the split lock ring is provided with a first slit, the split reinforcing ring is provided with a second slit aligned with the first slit of the split lock ring in a circumferential direction.

In other aspects, the plastic material is selected of polyethylene, polymethylmethacrylate (PMMA), polyetheretherketone (PEEK), polyetherketoneketone (PEKK), polyurethane and combination thereof, the split reinforcing ring is made of stainless steel, cobalt chrome alloys, zirconium, oxidized zirconium, tantalum, titanium alloys and combination thereof.

In other aspects, the outer shell is provided with a plurality of protrusions distributed in a circumferential direction on the second proximal inner surface portion and configured to prevent the inner liner from rotating relative to the outer shell.

In other aspects, the inner liner is provided with a plurality of recesses extending along a longitudinal axis on the outer surface of the inner liner.

In another aspect, a method for forming the bipolar prosthesis subassembly as described above is provided, the method comprises:
providing the outer shell, the inner liner, and the split lock ring;
inserting the split lock ring into the circumferential groove of the inner liner; and
inserting the inner liner into the outer shell, so as to form the bipolar prosthesis subassembly.

In other aspects, the method further comprises placing a split reinforcing ring into the circumferential groove of the split lock ring, before inserting the split lock ring into the circumferential groove of the inner liner.

In addition, since the outer shell, the inner liner, and the split lock ring are pre-formed as a bipolar prosthesis subassembly, the assembly process is easy. The only work is to insert a femoral head into the bipolar prosthesis subassembly along a longitudinal axis. When the femoral head is inserted, the split lock ring moves to the assembly position and is expanded so that the femoral head can be inserted into the sphere-shaped cavity. After the femoral head is inserted, the split lock ring elastically returns to the locking position due to its elastic property, so as to securely hold the femoral head in place inside the inner liner.

In addition, since the transition portion at the proximal end of the inner liner forms smooth transition to the sphere-shaped outer surface of the outer shell, the bipolar prosthesis assembly is presented as a sphere shape, which in turn protects the bone and tissue around the acetabulum during movement.

In addition, since the bipolar prosthesis subassembly includes two ring elements, i.e., the split lock ring and the split reinforcing ring, the split lock ring can be reinforced. Further areas of applicability of the present disclosure will become apparent from the detailed description, the claims and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings.

FIG. 1 schematically illustrates in perspective view an exemplary bipolar prosthesis subassembly in accordance with the present disclosure.

FIG. 2 schematically illustrates in a cross-section view an exemplary bipolar prosthesis subassembly in accordance with the present disclosure.

FIG. 3 schematically illustrates in perspective view an exemplary outer shell of the bipolar prosthesis subassembly in accordance with the present disclosure.

FIG. 4 schematically illustrates in another perspective view an exemplary outer shell of the bipolar prosthesis subassembly in accordance with the present disclosure.

FIG. 5 schematically illustrates in a cross-section view an exemplary outer shell of the bipolar prosthesis subassembly in accordance with the present disclosure.

FIG. 6 schematically illustrates in perspective view an exemplary inner liner of the bipolar prosthesis subassembly in accordance with the present disclosure.

FIG. 7 schematically illustrates in another perspective view an exemplary inner liner of the bipolar prosthesis subassembly in accordance with the present disclosure.

FIG. 8 schematically illustrates in a cross-section view an exemplary inner liner of the bipolar prosthesis subassembly in accordance with the present disclosure.

FIG. 9 schematically illustrates in a perspective view an exemplary lock ring of the bipolar prosthesis subassembly in accordance with the present disclosure.

FIG. 10 schematically illustrates in a cross-section view an exemplary lock ring of the bipolar prosthesis subassembly in accordance with the present disclosure.

FIG. 11 schematically illustrates in a plan view an exemplary lock ring of the bipolar prosthesis subassembly in accordance with the present disclosure.

FIG. 12 schematically illustrates in a perspective view an exemplary reinforcing ring of the bipolar prosthesis subassembly in accordance with the present disclosure.

### DETAILED DESCRIPTION OF EMBODIMENTS

As used herein, words such as “up”, “down”, “left”, and “right” used herein to define orientations generally refer to and are understood as orientations in association with the drawings and orientations in actual application. The term “longitudinal axis/direction” is defined relative to the direction along which the bipolar prosthesis subassembly is planted or removed, i.e., the direction along which the femoral head is inserted into the bipolar prosthesis subassembly. In addition, the term “distal” and/or “proximal” are defined relative to the longitudinal axis/direction. In addition, the term “equator” is defined as the largest diameter circle of the sphere-shaped cavity of the inner liner along the longitudinal axis/direction, which looks like the equator of the sphere-shaped cavity of the inner liner along the longitudinal axis/direction.

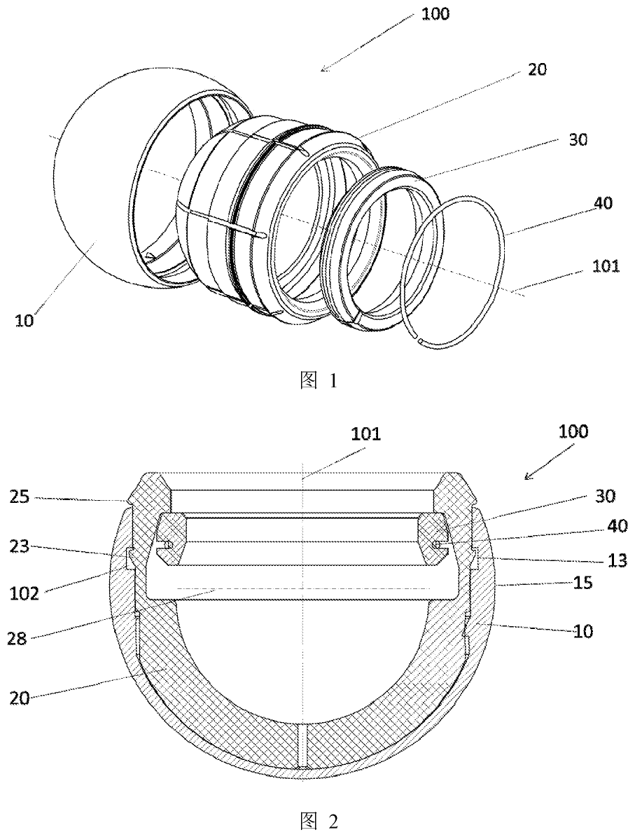

Referring now to the drawings, wherein like reference numbers refer to like features throughout the several views, FIG. 1 schematically illustrates in perspective view an exemplary bipolar prosthesis subassembly 100 in accordance with the present disclosure. FIG. 2 schematically illustrates in a cross-section view an exemplary bipolar prosthesis subassembly 100 in accordance with the present disclosure. The bipolar prosthesis assembly is generally used as a surgical implant to be implanted into the acetabulum of a patient to replace the femoral head of the hip joint. As used herein, the term “bipolar” is defined since there are two bearing surfaces after implant of the bipolar prosthesis assembly, i.e., one bearing surface between the outer shell and the acetabulum and the other bearing surface between the femoral head and the inner liner.

As a non-limiting example, the bipolar prosthesis subassembly 100 may comprises an outer shell 10, an inner liner 20, and a split lock ring 30. Optionally, the bipolar prosthesis subassembly 100 may also comprises a split reinforcing ring 40.

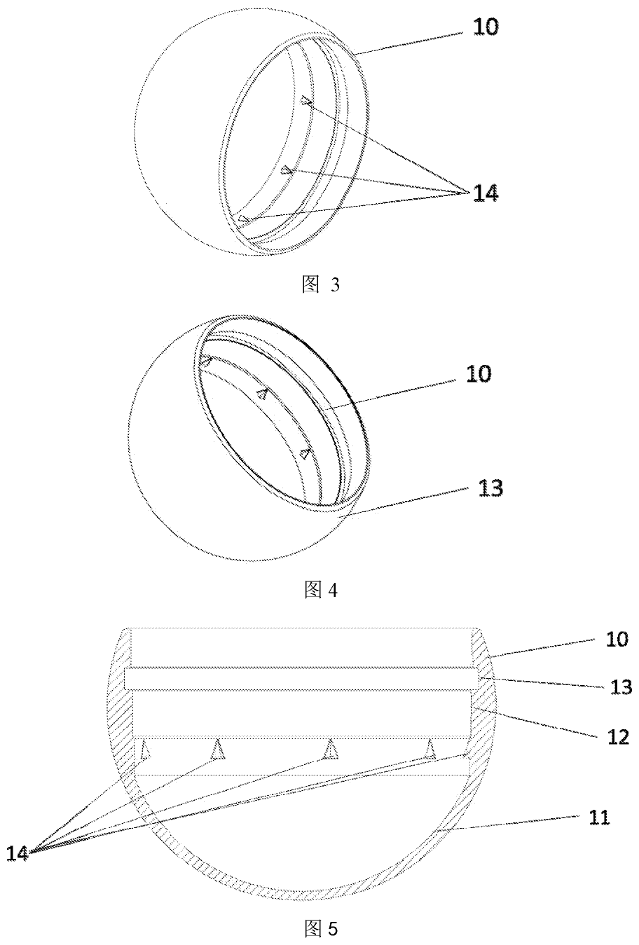

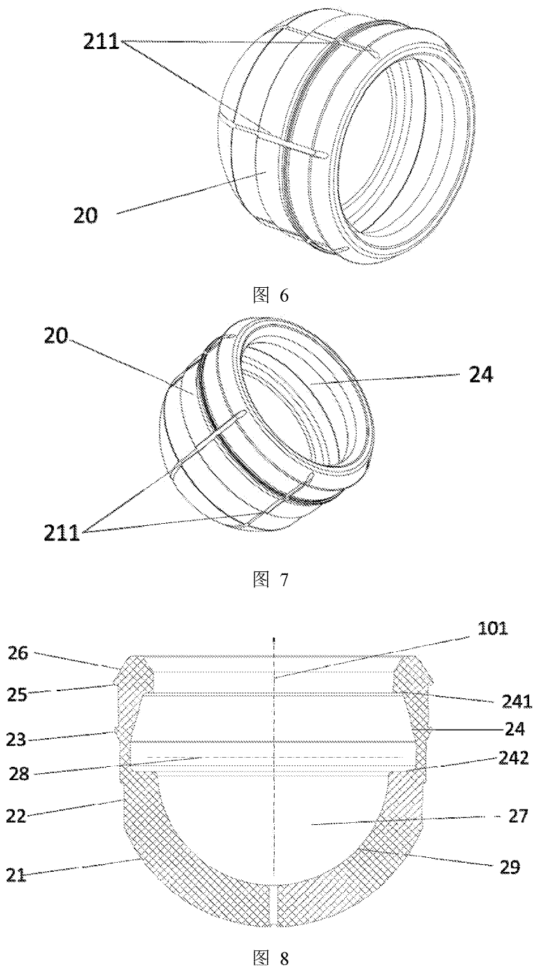

FIG. 3 schematically illustrates in perspective view an exemplary outer shell 10 of the bipolar prosthesis subassembly 100 in accordance with the present disclosure. FIG. 4 schematically illustrates in another perspective view an exemplary outer shell 10 of the bipolar prosthesis subassembly 100 in accordance with the present disclosure. FIG. 5 schematically illustrates in a cross-section view an exemplary outer shell 10 of the bipolar prosthesis subassembly 100 in accordance with the present disclosure. As a non-limiting example, the outer shell 10 may include a sphere-shaped outer surface 15 and an inner surface with a first distal inner surface portion 11 of sphere-shape and a second proximal inner surface portion 12 of cylindrical shape.

As a non-limiting example, the outer shell 10 may be provided with a circumferential locking groove 13 on the second proximal inner surface portion 12. Optionally, the outer shell 10 may also include a plurality of protrusions 14. The protrusions 14 are distributed in a circumferential direction on the second proximal inner surface portion 12 and configured to prevent the inner liner 20 from rotating relative to the outer shell 10. As a non-limiting example, the outer shell 10 may include nine protrusions 14. However, as will be understood by those skilled in the art, the outer shell 10 may include any other suitable number of protrusions 14, such as, 3, 4, 5, 6 or like, without departing the scope of the disclosure. As a non-limiting example, the outer shell 10 may be made from CoCr alloy, for example CoCrMo. However, as will be understood by those skilled in the art, the outer shell 10 may made from any suitable biocompatible material, such as stainless steel, cobalt chrome alloys, zirconium, oxidized zirconium, tantalum, titanium alloys and combination thereof, without departing the scope of the disclosure. As a non-limiting example, the outer shell 10 may be made by casting. However, as will be understood by those skilled in the art, the outer shell 10 may made from any suitable method, such as 3D printing, without departing the scope of the disclosure.

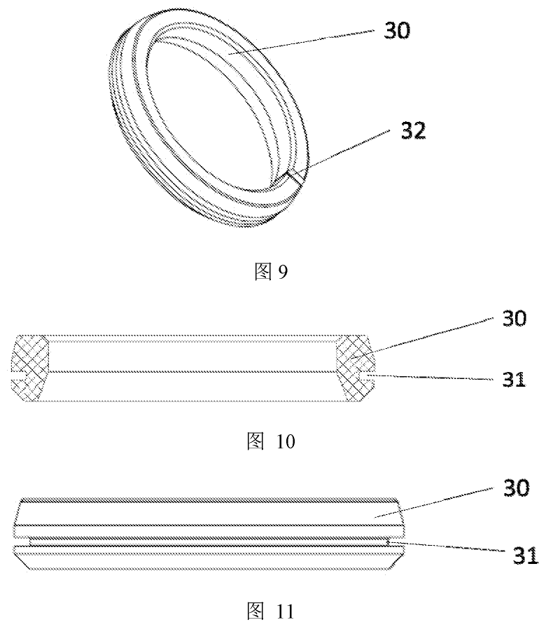

FIG. 6 schematically illustrates in perspective view an exemplary inner liner 20 of the bipolar prosthesis subassembly 100 in accordance with the present disclosure. FIG. 7 schematically illustrates in another perspective view an exemplary inner liner 20 of the bipolar prosthesis subassembly 100 in accordance with the present disclosure. FIG. 8 schematically illustrates in a cross-section view an exemplary inner liner 20 of the bipolar prosthesis subassembly 100 in accordance with the present disclosure. The inner liner 20 is configured to dispose inside of the outer shell 10 in a manner of non-interference fit. Since the inner liner 20 is disposed inside of the outer shell 10 in a manner of non-interference fit, the inner liner 20 may not be deformed during the assembly of the bipolar prosthesis subassembly 100 and during the use of the bipolar prosthesis assembly, thus ensuring the dimensional accuracy and roundness of the inner liner 20, which in turn reduces the wear between the inner liner 20 and the femoral head.

As a non-limiting example, the inner liner 20 may include a sphere-shaped inner surface 29 which is configured to house an artificial femoral head. The sphere-shaped inner surface 29 of the inner liner 20 defines a sphere-shaped cavity 27 defining an equator 28, that is, the largest diameter circle of the sphere-shaped cavity 27 of the inner liner 20 along the longitudinal axis 101. As a non-limiting example, the inner liner 20 may also include an outer surface with a first distal outer surface portion 21 of sphere-shape and a second proximal outer surface portion 22 of cylindrical shape. The first distal outer surface portion 21 and the second proximal outer surface portion 22 of the inner liner 20 correspond to the first distal inner surface portion 11 and the second proximal inner surface portion 12 of the outer shell 10, respectively.

As a non-limiting example, the inner liner 20 may include a circumferential distal locking protrusion 23. The circumferential distal locking protrusion 23 may be configured to snap into the circumferential locking groove 13 of the outer shell 10 to secure the inner liner 20 within the outer shell 10.

As shown in FIG. 2, the circumferential distal locking protrusion 23 is sized less than the circumferential locking groove 13 of the outer shell 10, so as to form a space 102 between the circumferential distal locking protrusion 23 and the circumferential locking groove 13 of the outer shell 10. As a non-limiting example, the space 102 may extend along the longitudinal axis 101 between the distal end of the circumferential distal locking protrusion 23 and the distal end of the circumferential locking groove 13 of the outer shell 10. Optionally, the space 102 may be present between the radially outer surface of the circumferential distal locking protrusion 23 and the radially inner surface of the circumferential locking groove 13 of the outer shell 10. As another option, the space 102 may be present both between the distal end of the circumferential distal locking protrusion 23 and the distal end of the circumferential locking groove 13 of the outer shell 10 as well as between the radially outer surface of the circumferential distal locking protrusion 23 and the radially inner surface of the circumferential locking groove 13 of the outer shell 10. Due to the second proximal outer surface portion 22 of cylindrical shape and the second proximal inner surface portion 12 of cylindrical shape as well as the existence of the space 102, the inner liner 20 may not be deformed during the assembly of the bipolar prosthesis subassembly 100 and during the use of the bipolar prosthesis assembly, thus ensuring the dimensional accuracy and roundness of the inner liner 20, which in turn reduces the wear between the inner liner 20 and the femoral head.

As a non-limiting example, the inner liner 20 may include a circumferential groove 24 on the inner surface 29. The circumferential groove 24 of the inner liner 20 extends from a first position 241 below and in proximity to the equator 28 to a second position 242 above the equator 28 and tapers from the first position 241 to the second position 242.

As a non-limiting example, the second proximal outer surface portion 22 of the inner liner 20 may extend beyond the sphere-shaped outer surface 15 of the outer shell 10 along a proximal direction. In this regard, the inner liner 20 may be provided with a partial sphere-shaped transition portion 26 at the proximal end thereof, which forms smooth transition to the sphere-shaped outer surface 15 of the outer shell 10. The transition portion 26 includes a rounded protrusion 25 adjacent to the outer shell 10, which forms smooth transition to the sphere-shaped outer surface 15 of the outer shell 10. Since the arc-shaped transition position at the proximal end of the inner liner 20 forms smooth transition to the sphere-shaped outer surface 11 of the outer shell 10, the bipolar prosthesis assembly is presented as a sphere shape, which in turn protects the bone and tissue around the acetabulum during movement.

As a non-limiting example, the inner liner 20 may also include a plurality of recesses 211 extending along a longitudinal axis 101 on the outer surface of the inner liner 20. As a non-limiting example, the inner liner 20 may include six recesses 211. However, as will be understood by those skilled in the art, the inner liner 20 may include any other suitable number of recesses 211, such as, 3, 4, 5, 7, 8 or like, without departing the scope of the disclosure. As a non-limiting example, the inner liner 20 may be made from polyethylene, for example ultra-high molecular weight polyethylene. However, as will be understood by those skilled in the art, the inner liner 20 may made from any suitable biocompatible material, such as polymethylmethacrylate (PMMA), polyetheretherketone (PEEK), polyetherketoneketone (PEKK), polyurethane and combination thereof, without departing the scope of the disclosure. The polymer materials may also be reinforced with carbon, metal, or glass or any other suitable material. As a non-limiting example, the inner liner 20 may be made by inject molding. However, as will be understood by those skilled in the art, the inner liner 20 may made from any suitable method, such as 3D printing, without departing the scope of the disclosure.

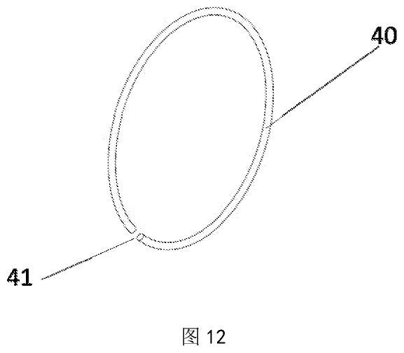

FIG. 9 schematically illustrates in a perspective view an exemplary lock ring 30 of the bipolar prosthesis subassembly 100 in accordance with the present disclosure. FIG. 10 schematically illustrates in a cross-section view an exemplary lock ring 30 of the bipolar prosthesis subassembly 100 in accordance with the present disclosure. FIG. 11 schematically illustrates in a plan view an exemplary lock ring 30 of the bipolar prosthesis subassembly 100 in accordance with the present disclosure. The split lock ring 30 may be configured to elastically snap into the circumferential groove 24 of the inner liner 20. As a non-limiting example, the split lock ring 30 is movable in the circumferential groove 24 between an assembly position and a locking position along the longitudinal axis. In the assembly position, the split lock ring 30 is located at the equator 28 and the center of the split lock ring 30 is located slightly above the equator 28, so that once the femoral head is inserted into the sphere-shaped cavity 27, the split lock ring 30 will automatically returns to the locking position due to its elastics and the tapering outer shape of the femoral head above the equator 28. The split lock ring 30 thus can be expanded to an inner diameter lager than an outer diameter of a femoral head so that the femoral head can be inserted into the sphere-shaped cavity 27. In the locking position, the split lock ring 30 elastically returns to an inner diameter less than the outer diameter of the femoral head so as to securely hold the femoral head in place inside the inner liner 20. After the femoral head is inserted into the sphere-shaped cavity 27, the split lock ring (30) elastically returns to the locking position. Since the outer shell 10, the inner liner 20, and the split lock ring 30 are pre-formed as a bipolar prosthesis subassembly 100, the assembly process is easy. The only work is to insert a femoral head into the bipolar prosthesis subassembly 100 along a longitudinal axis A. When the femoral head is inserted, the split lock ring 30 moves to the assembly position and is expanded so that the femoral head can be inserted into the sphere-shaped cavity 27. After the femoral head is inserted, the split lock ring 30 elastically returns to the locking position due to its elastic property, so as to securely hold the femoral head in place inside the inner liner 20.

FIG. 12 schematically illustrates in a perspective view an exemplary reinforcing ring 40 of the bipolar prosthesis subassembly 100 in accordance with the present disclosure.

As shown in FIG. 9 and FIG. 10, the split lock ring 30 is provided with a circumferential groove 31 at outer surface thereof. The split reinforcing ring 40 elastically snaps into the circumferential groove 31 of the split lock ring 30. The split lock ring 30 includes a first slit 32, and the split reinforcing ring 40 is provided with a second slit 41 aligned with the first slit 32 of the split lock ring 30 in a circumferential direction. In this regard, since the bipolar prosthesis subassembly 100 includes two ring elements, i.e., the split lock ring 30 and the split reinforcing ring 40, the split lock ring 30 can be reinforced.

As a non-limiting example, the split lock ring 30 may be made from polyethylene, for example ultra-high molecular weight polyethylene. However, as will be understood by those skilled in the art, the split lock ring 30 may made from any suitable biocompatible material, such as polymethylmethacrylate (PMMA), polyetheretherketone (PEEK), polyetherketoneketone (PEKK), polyurethane and combination thereof, without departing the scope of the disclosure. The polymer materials may also be reinforced with carbon, metal, or glass or any other suitable material. As a non-limiting example, the split lock ring 30 may be made by inject molding. However, as will be understood by those skilled in the art, the split lock ring 30 may made from any suitable method, such as 3D printing, without departing the scope of the disclosure.

As a non-limiting example, the split reinforcing ring 40 may be made from CoCr alloy, for example CoCrMo. However, as will be understood by those skilled in the art, the split reinforcing ring 40 may made from any suitable biocompatible material, such as stainless steel, cobalt chrome alloys, zirconium, oxidized zirconium, tantalum, titanium alloys and combination thereof, without departing the scope of the disclosure. As a non-limiting example, the split reinforcing ring 40 may be made by casting. However, as will be understood by those skilled in the art, the split reinforcing ring 40 may made from any suitable method, such as 3D printing, without departing the scope of the disclosure.

Aspects of the present disclosure have been described in detail with reference to the illustrated embodiments; those skilled in the art will recognize, however, that many modifications may be made thereto without departing from the scope of the present disclosure. The present disclosure is not limited to the precise construction and compositions disclosed herein; any and all modifications, changes, and variations apparent from the foregoing descriptions are within the scope of the disclosure as defined by the appended claims. Moreover, the present concepts expressly include any and all combinations and subcombinations of the preceding elements and features.

1. A bipolar prosthesis subassembly comprising:
an outer shell;
an inner liner configured to dispose inside of the outer shell in a manner of non-interference fit, the inner liner is provided with a circumferential groove; and
a split lock ring configured to elastically snap into the circumferential groove of the inner liner,
wherein the inner liner defines a sphere-shaped cavity defining an equator, the circumferential groove of the inner liner extends from a first position below and in proximity to the equator to a second position above the equator and tapers from the first position to the second position, the split lock ring is movable in the circumferential groove between an assembly position and a locking position along the longitudinal axis, in the assembly position, the split lock ring can be expanded to an inner diameter lager than an outer diameter of a femoral head so that the femoral head can be inserted into the sphere-shaped cavity, in the locking position, the split lock ring elastically returns to an inner diameter less than the outer diameter of the femoral head so as to securely hold the femoral head in place inside the inner liner.

2. The bipolar prosthesis subassembly according to claim 1, wherein the outer shell includes a sphere-shaped outer surface and an inner surface with a first distal inner surface portion of sphere-shape and a second proximal inner surface portion of cylindrical shape, the inner liner includes a sphere-shaped inner surface and an outer surface with a first distal outer surface portion of sphere-shape and a second proximal outer surface portion of cylindrical shape.

3. The bipolar prosthesis subassembly according to claim 2, wherein the outer shell is provided with a circumferential locking groove on the second proximal inner surface portion, the inner liner is provided with a circumferential distal locking protrusion configured to snap into the circumferential locking groove of the outer shell to secure the inner liner within the outer shell.

4. The bipolar prosthesis subassembly according to claim 3, wherein the circumferential distal locking protrusion is sized less than the circumferential locking groove of the outer shell, so as to form a space between the circumferential distal locking protrusion and the circumferential locking groove of the outer shell.

5. The bipolar prosthesis subassembly according to claim 4, wherein the second proximal outer surface portion of the inner liner extends beyond the sphere-shaped outer surface of the outer shell along a proximal direction, and is provided with an arc-shaped transition position at the proximal end thereof, which forms smooth transition to the sphere-shaped outer surface of the outer shell.

6. The bipolar prosthesis subassembly according to claim 1, wherein the split lock ring provided with a circumferential groove at outer surface thereof, the bipolar prosthesis subassembly further comprising a split reinforcing ring configured to elastically snap into the circumferential groove of the split lock ring.

7. The bipolar prosthesis subassembly according to claim 6, wherein the split lock ring is made of a plastic material, the split reinforcing ring is made of a metal or metal alloy.

8. The bipolar prosthesis subassembly according to claim 7, wherein the split lock ring is provided with a first slit, the split reinforcing ring is provided with a second slit aligned with the first slit of the split lock ring in a circumferential direction.

9. The bipolar prosthesis subassembly according to claim 8, wherein the plastic material is selected of polyethylene, polymethylmethacrylate (PMMA), polyetheretherketone (PEEK), polyetherketoneketone (PEKK), polyurethane and combination thereof, the split reinforcing ring is made of stainless steel, cobalt chrome alloys, zirconium, oxidized zirconium, tantalum, titanium alloys and combination thereof.

10. The bipolar prosthesis subassembly according to claim 1, wherein the outer shell is provided with a plurality of protrusions distributed in a circumferential direction on the second proximal inner surface portion and configured to prevent the inner liner from rotating relative to the outer shell.

11. The bipolar prosthesis subassembly according to claim 1, wherein the inner liner is provided with a plurality of recesses extending along a longitudinal axis on the outer surface of the inner liner.

12. A method for forming the bipolar prosthesis subassembly according to claim 1, the method comprising:
providing the outer shell, the inner liner, and the split lock ring;
inserting the split lock ring into the circumferential groove of the inner liner; and
inserting the inner liner into the outer shell, so as to form the bipolar prosthesis subassembly.

13. The method for forming the bipolar prosthesis subassembly according to claim 12, further comprising:
placing a split reinforcing ring into the circumferential groove of the split lock ring, before inserting the split lock ring into the circumferential groove of the inner liner.
